SEQUENCE LISTING (2 sequences)

SEQ ID NO: 1 - DNA, 20 bp, Murine Melanoma B-16
gacctcagttccccttcaaa

SEQ ID NO: 2 - DNA, 20 bp, Murine Melanoma B-16
tctcatccccagttagttct

(19) United States
(12) Patent Application Publication
(10) Pub. No.: US 2006/0216253 A1
(43) Pub. Date: Sep. 28, 2006

(54) Whitening cosmetics containing morus alba extracts

(75) Inventors: Jeong-Ha Kim, North Brunswick, NJ (US); Mingfu Wang, Belle Mead, NJ (US); James Simon, Princeton, NJ (US); Abraham Kim, Morganville, NJ (US)

Correspondence Address: Altimatia, L.L.C, 16 Elm Ridge Road, Pennington, NJ, 08534, US

(73) Assignee: Time and Cross, Inc., North Brunswick, NJ (US)

(21) Appl. No.: 11/347,884

(22) Filed: Feb. 6, 2006

Related U.S. Application Data
(60) Provisional application No. 60/664,685, filed on Mar. 23, 2005.

(51) Int. Cl. A61K 8/97

(52) U.S. Cl. 424/62; 424/769

(57) ABSTRACT

The present invention relates to cosmetic products, comprising extracts of Morus alba, which have a whitening effect. According to the present invention extracting powdered Morus alba twig with aqueous ethanol provides an extract comprising oxyresveratrol and mulberroside A. Extracts enriched in oxyresveratrol or mulberroside A, as well as purified oxyresveratrol and mulberrocide A may be also be obtained. The extracts or purfied compounds may be added to conventional skin care cosmetics to give whitening cosmetics.

### CROSS REFERENCE TO RELATED APPLICATIONS

This application claims the benefit of U.S. Provisional Patent Application Ser. No. 60/664,685.

### FIELD

The present invention concerns extracts and compounds isolated from Morus alba, commonly known as mulberry, as well as their isolation and use as tyrosinase inhibitors, including attendant applications thereof, including the inhibition of melanogenesis in humans, animals, and plants.

### BACKGROUND

All living organisms exhibit a variety of responses against the external environment to maintain the homeostasis of life. The synthesis of melanin also relates to this phenomenon. In general, there are various reasons for the darkening of skin color, ultraviolet ray being the principal. When skin is exposed to ultraviolet ray, melanin is synthesized in melanocytes, a type of skin cell, and released resulting in a darkening of the skin. Melanin pigmentation in human skin is a major defense mechanism against ultraviolet light from the sun [Pawelek, et al. (1992) Cosmetics & Toiletries 107, 61-68], but abnormal pigmentation such as freckles or chloasma (liver spot, melasma) can be a serious aesthetic problem [Sugai T., (1992) Hifu. Skin Res. 34, 522-529; Mishima Y. et al (1983) J. Invest. Dermatol. 81, 106-114]. Studies by others are being conducted to discover melanogenic inhibitory compounds to prevent or to cure these hyperpigmentary disorders. Nearly all studies are mainly concentrated on searching for materials that have inhibitory activities on tyrosinase, a key enzyme for melanin biosynthesis.

Tyrosinase and other enzymes, such as tyrosinase related protein 1 (TRP-1) and tyrosinase related protein 2 (TRP-2, Dopachrome tautomerase), are responsible for the biosynthesis of melanin [P. Aroca et al (1993) J. Biol. Chem. 268, 25650]. Among these enzymes, tyrosinase plays the most important role in melanin synthesis. Tyrosinase is a copper-containing enzyme and is exclusively expressed in melanocytes, the melanin producing cells in the epidermis. Tyrosinase is capable of producing melanin pigments by itself, without the assistance of other melanogenic enzymes. Fibroblast cells transfected with tyrosinase cDNA produced melanin in their lysosome-like structures. [Bouchard et al (1989) J. Exp. Med. 169, 2029-2042; Kondoh et al (1993) J. Invest. Dermatol. 101, 394A; Winder et al (1993) J. Cell Sci. 104, 467-475] The regulation of tyrosinase expression is not fully understood. An understanding of the mechanism of melanin synthesis is thus central to the development of whitening agents or cosmetic products having a whitening effect. To prevent darkening of the skin, the first step is to inhibit the process of generating melanin. Such an inhibition can be performed by inhibiting the activity of tyrosinase.

An understanding of the mechanism of melanin synthesis is very important to the development of whitening agents or cosmetic products having whitening effect. The interpretation of the melanin-control mechanism may provide a method of preventing color deposition by melanin. In the process of melanogenesis in melanocytes, the tyrosinase enzyme acts on its substrate tyrosine to yield dopaquinone which subsequently and sequentially oxidizes to dopachrome, 5,6-dihydroxyindole (DHI), 5,6-dihydroxyindol-2-carboxylic acid (DHICA), and monomers of melanin, which polymerize to form the co-polymeric black pigment, melanin.

The mode of action of most whitening agents is via their inhibitory effect on tyrosinase, yet the mechanism of melanogenesis is complicated and remains unclear. At present, research to develop agents with tyrosinase-inhibiting activity is intensive. Representative tyrosinase inhibitors include kojic acid, arbutin, hydroquinone, ascorbic acid (vitamin C), and licorice extract. Among these, kojic acid forms a chelate with a copper ion at the active site of tyrosinase to inhibit the enzyme activity. Although possessing strong inhibitory activity on tyrosinase, it suffers from stability problems during cosmetic formulation and irritates the skin. Furthermore, the use of hydroquinone in cosmetic products is now prohibited in several countries. Ascorbic acid and its derivatives suffer from chemical instability, thus rendering these compounds relatively ineffective as practical skin whiteners. Further background information may be found in U.S. Pat. Nos. 5,872,254, 6,071, 525 and 6,197,304. These patents and other references cited within this application are hereby incorporated herein by reference. Materials extracted from various plants exhibit whitening effects, but most such materials have substantial inhibitory effects on tyrosinase activity only when used in high concentrations (IC₅₀ values of G. glabra, Rh. Palmatum, S. japonica, Trichosanthes kirilowii, and Lycium chinensis were determined as 71.0, 86.3, 93.4, 155.6 and 297.6 μg/mL). When used in relatively low concentrations, tyrosinase inhibition activity is hardly detectable. Accordingly, the use of such materials as whitening agents is impractical. Thus, there remains a need in the art for tyrosinase inhibitors possessing sufficient activity and stability for their effective use in cosmetic applications for whitening of the skin.

### SUMMARY

Disclosed are whitening cosmetics containing one or more compounds from an extract of Morus alba, commonly known as mulberry. We have isolated oxyresveratrol and mulberroside A from mulberry, and have found these compounds as well as extracts containing them to be potent and long-lasting inhibitors of tyrosinase activity. We dried and powdered twigs of Morus alba, subjected the material to solvent extraction, and distilled under reduced pressure to obtain extracts from Morus alba which can be added to cosmetics to form compositions for skin whitening. The compounds oxyresveratrol and mulberroside A were purified from the extract of Morus alba via solvent fractionation and column chromatography. The whitening cosmetics containing the extracts from Morus alba, oxyresveratrol and mulberroside A, respectively, of the present invention exhibit excellent whitening effects and have been determined to be strong inhibitors of melanogenesis.

### BRIEF DESCRIPTION OF THE DRAWINGS

FIG. 1 is a graph of % inhibition of Mushroom Tyrosinase activity versus concentration of oxyresveratrol and mulberroside A of the present invention as well as the reference compounds arbutin and kojic acid.

FIG. 2 is a graph of % inhibition of Mushroom Tyrosinase activity versus concentration of Morus alba extract of the present invention as well as the reference compound kojic acid.

FIG. 3 is a graph of % inhibition of Mushroom Tyrosinase activity versus reaction time for Morus alba extract, oxyresveratrol, and mulberroside A of the present invention as well as the reference compounds arbutin and kojic acid.

FIG. 4 is a photograph of a microtiter plate showing the effects of various concentrations of Morus alba extract, oxyresveratrol, and mulberroside A of the present invention as well as the reference compounds arbutin and kojic acid on the activity of Mushroom Tyrosinase after incubation for 24 hours.

FIG. 5 is a photograph of an electrophoresis gel (A) and a graph (B) depicting the inhibition of tyrosinase mRNA expression in murine melanoma B-16 cells effected by Morus alba extract and mulberroside A of the present invention.

### DETAILED DESCRIPTION

Preparation of Morus alba Extract:

Morus alba extracts can be prepared from twigs of Morus alba. First, the Morus alba twigs are ground to a fine powder, which is then subjected to extraction with 30-100% ethanol:water, preferably 70% ethanol:water. When we subjected powdered Morus alba twigs to extraction with 70% ethanol:water, we obtained a light-yellow solution separately from an insoluble fraction. Evaporation of the light-yellow solution provided a yellow solid that amounted to approximately 10% by weight of the Morus alba twig. This solid extract typically contains 0.5-5% oxyresveratrol and 0.5-5% of mulberroside A.

Preparation of Oxyresveratrol-Enriched and Mulberroside A-Enriched Extracts:

An oxyresveratrol-enriched extract and a mulberroside A-enriched extract can be prepared from the Morus alba extract by column chromatography (silica gel, reverse-phase silica gel, Polyamide-6, Sephedex LH-20, ion-exchange column chromatography), preferably polyamide-6 column chromatography or solvent partitioning. To purify the compounds for analysis of inhibitory activity on various tyrosinases, we used a polyamide column. We dissolved 20 g of Morus alba extract in 200 mL methanol/water (40:60, v/v) and directly loaded the sample to a (30 g) polyamide-6 column. The column was first eluted with 600 mL of methanol/water (40:60, v/v) to obtain a yellow-red liquid, an extract enriched in mulberroside A. After washing the column with 500 mL of methanol/water (70:30, v/v) an oxyresveratrol-enriched liquid extract was obtained via elution with 500 mL of pure methanol. The respective liquid extracts were evaporated to dryness under reduced pressure to obtain the mulberroside A-enriched and oxyresveratrol-enriched extracts.

To prepare the enriched extracts by solvent partitioning, 10 g of Morus alba extract was suspended into 200 g of water and partitioned with chloroform (3×200 mL). The aqueous layer was then extracted with ethyl acetate (3×200 mL). The ethyl acetate fraction was evaporated to dryness under reduced pressure to obtain an oxyresveratrol-enriched extract, and the aqueous fraction was evaporated to dryness to obtain a mulberroside A-enriched extract. The oxyresveratrol-enriched extract typically contains 2-50% oxyresveratrol and the mulberroside A-enriched extract typically contains 2-50% of mulberroside A.

Isolation of Oxyresveratrol and Mulberroside A from Enriched Extracts:

We purified oxyresveratrol and mulberroside A from the respective enriched extracts using thin layer chromatography (TLC), silica gel column chromatography, and Sephadex LH-20 gel filtration chromatography. The structures of these two compounds were confirmed by MS and NMR spectra. For example, to prepare pure compound at lab scale, 6 g of oxyresveratrol-enriched extract was loaded directly onto a Sephadex LH-20 column (120 g of Sephadex LH-20 gel) and eluted with pure methanol to obtain four subfractions, the last of which was evaporated to dryness and then subjected to chromatography on a silica gel column with 6:1:1 chloroform-methanol-water as the mobile phase to get 1 g of pure oxyresveratrol. 20 g of mulberroside A-enriched extract was loaded directly onto a silica gel column (300 g of silica gel) and eluted with 2.5:1:0.1 chloroform-methanol-water to obtain five subfractions, the last of which was subjected to Sephadex LH-20 column chromatography to obtain 700 mg of pure mulberroside A.

We have determined that Morus alba extract, as well as purified oxyresveratrol and mulberroside A, possess a variety of biological activities that correlate with a variety of applications. Specifically, these activities include anti-browning effects for plants and skin whitening effects as evidenced by the inhibitory effects of these agents on mushroom tyrosinase, murine tyrosinase and human tyrosinase, on melanin production in the melanoma B-16 and melan-a cell lines in culture, and on tyrosinase mRNA expression. Moreover, the extracts and compounds of the present invention exhibit inhibition of tyrosinase activity for a much longer duration than whitening agents of the prior art, such as arbutin and kojic acid. Thus, the extracts and compounds of the present invention are well-suited for use as whitening agents in various cosmetic applications.

Inhibitory effects on Tyrosinase Activity:

Inhibitory Effects on Mushroom Tyrosinase

We have analyzed the inhibitory activity of the extracts and compounds of the present invention on tyrosinase isolated from mushrooms (Sigma Co. USA). L-Tyrosine, the substrate for tyrosinase, was dissolved in 50 mM sodium phosphate buffer (pH6.8) to prepare a solution of 0.1 mg/mL L-tyrosine. Each of the whitening agents assayed, namely, the agents of the present invention as well as the prior art whitening agents kojic acid and arbutin, which were used as references, was dissolved and diluted in dimethyl sulfoxide (DMSO) to a concentration of 1 mg/mL, the stock solution for preparation of “test samples” for all of the experiments described herein. 100 μL of the L-tyrosine solution was introduced into a test tube, and 100 μL of 50 mM sodium phosphate buffer (pH6.8) with or without test samples were added thereto, and then 10 μL of 200 U/mL tyrosinase were mixed and incubated at 37° C. for 15 min.

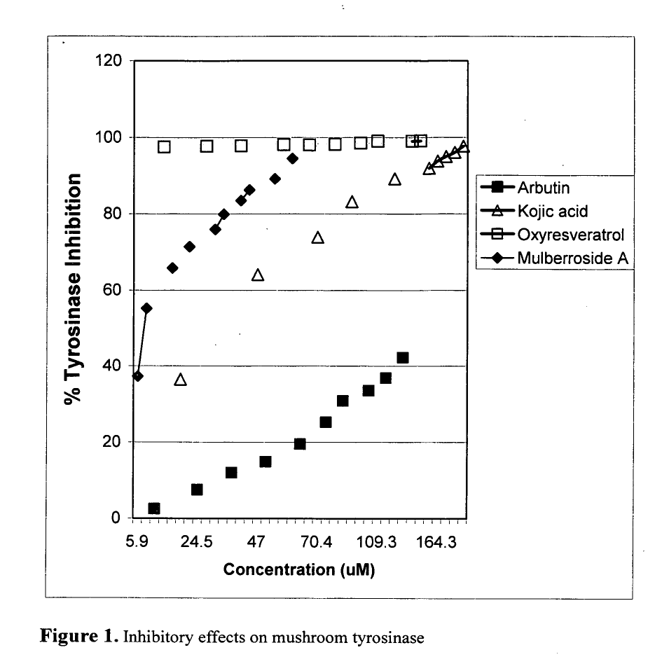

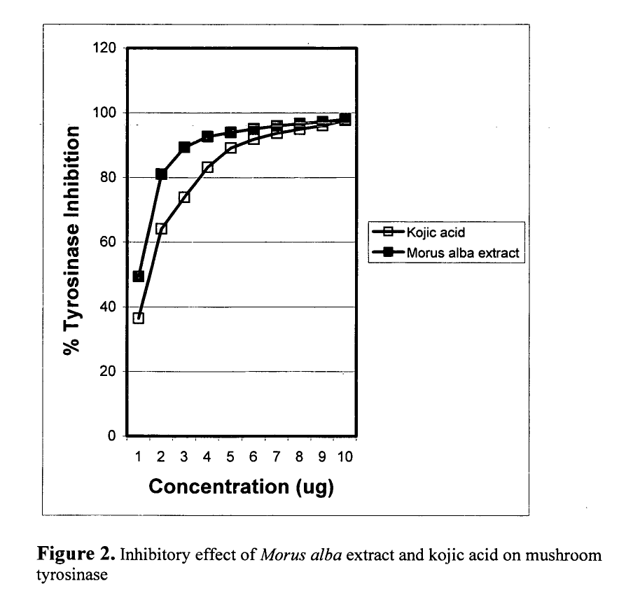

The amount of dopachrome in the reaction mixture was determined spectrophotmetrically using absorbance measurement at 490 nm. Results are shown in FIGS. 1 and 2. Based on the optical density at 490 nm (OD₄₉₀), the inhibitory activity of the sample was expressed as the concentration which inhibits 50% of the enzyme activity (IC₅₀). The results are shown in Table 1.

All of the agents tested exhibited dose-dependent inhibitory effects on mushroom tyrosinase activity. Among the agents tested, oxyresveratrol and mulberroside A were the most potent inhibitors of mushroom tyrosinase. Morus alba extract showed stronger inhibitory activity on mushroom tyrosinase than kojic acid, as IC₅₀ values for Morus alba extract and kojic acid were 1.3 μg/mL and 1.49 μg/mL, respectively.

Inhibitory Effects on Murine Tyrosinase

Murine tyrosinase was prepared from the melanoma B-16 cell line. Murine melanoma B-16 cells were grown in DMEM (13.4 mg/mL of Dulbecco's modified Eagle's medium, 24 mM NaHCO3, 10 mM HEPES, 143 units/mL of penicillin G potassium, 160 μg/mL of streptomycin sulfate, pH 7.1) containing 10% fetal bovine serum (FBS) with 5% CO₂ at 37° C. When the cells were confluent, melanoma cells were removed from culture dishes and washed once with PBS buffer. The cell suspension was centrifuged at 250×g for 10 min at 4° C. The cell pellet was suspended in 0.5 mL of 50 mM sodium phosphate buffer (pH 6.8) containing 0.1 mM phenylmethylsulfonyl fluoride and 0.5% Triton X-100. The cell suspension was sonicated six times for 30 sec each with 1 min intervals and then incubated at 4° C. for 1 hr to solubilize the tyrosinase. After centrifugation at 50,000×g for 20 min at 4° C., supernatant was dialyzed against 50 mM sodium phosphate buffer (pH 6.8) and then used as a source of murine tyrosinase.

As in the mushroom tyrosinase experiment discussed above, the amount of dopachrome in the reaction mixture was determined spectrophotmetrically using absorbance measurement at 490 nm. IC₅₀ values for the inhibition of murine tyrosinase activity by the agents assayed are shown in Table 1. The agents of the present invention were found to be more potent inhibitors of murine tyrosinase than either of the prior art agents kojic acid or arbutin.

Inhibitory Effects on Human Tyrosinase

Human tyrosinase was prepared from the HM3KO human melanoma cell line. HM3KO cells were cultured in modified Eagles medium supplemented with 10% fetal calf serum. Cultured cells were removed from culture dishes and washed once with PBS buffer and centrifuged. The cell pellet was sonicated in 100 mM sodium phosphate buffer (pH6.8) containing 0.1 mM phenylmethylsulfonyl fluoride (PMSF) and 1% Triton X-100, incubated in ice for 1 hr to solubilize the tyrosinase, and centrifuged. The supernatant was dialyzed against 50 mM sodium phosphate buffer (pH 6.8) and then used as the source of human tyrosinase for use in our inhibition assays.

As in the mushroom and murine tyrosinase experiments discussed above, the amount of dopachrome in the reaction mixture was determined spectrophotmetrically using absorbance measurement at 490 nm. IC₅₀ values for the inhibition of human tyrosinase activity by the agents assayed are shown in Table 1. The agents of the present invention were found to be more potent inhibitors of human tyrosinase than either of the prior art agents kojic acid or arbutin.

TABLE 1
Inhibitory effects on mushroom, murine and human tyrosinases

| | Mushroom | Murine | Human |
|---|---|---|---|
| | Tyrosinase | Tyrosinase | Tyrosinase |
| Compounds | IC₅₀ (μM) | IC₅₀(μM) | IC₅₀(μM) |
| Arbutin | 144.6 | >500 | >500 |
| Kojic acid | 34.7 | 402 | 421 |
| Morus alba | 1.3 | 39.5 | 47.2 |
| extract* | | | |
| Oxyresveratrol | 2.4 | 42.5 | 57.4 |
| Mulberroside A | 9.5 | 61.5 | 76.8 |

*Morus alba extract: μg/mL

Effect on Melanogenesis and Cell Viability

Murine Melanoma B-16

We assayed the agents of the present invention as well as the reference compounds arbutin and kojic acid to determine their effects on melanogenesis and cell viability in a commercially available murine cell line, namely, the B-16 melanoma (ATCC CRL6323) cell line. The melanoma cell line was grown in DMEM culture medium containing 4.5 g of glucose/l, 10% fetal bovine serum and 1% antibiotic agent and cultivated at 37° C. under a condition of 5% CO₂. The cell suspension was inoculated in a 50 mL T-flask (3×10⁵ cells/mL) and the cells were allowed to completely adhere to the plate for 24 hr. Then, test samples diluted in DMEM medium at concentrations of 1, 5, 10, 50, and 100 μg/mL were added to the cultivated cells, and the mixture was cultivated at 5% CO₂ and 37° C. for 3 days. After cultivation, culture medium was thoroughly removed and the cells were isolated via trypsin treatment. The suspensions were then centrifuged for 5 min to collect cells. The obtained cells were treated with 5% trichloroacetic acid (TCA), stirred and centrifuged. The precipitated melanin was washed with phosphate buffered saline (PBS), and treated with 1 N NaOH to dissolve the melanin therein. Absorbance at 475 nm was measured. Melanin concentration was determined from a standard concentration curve of synthetic melanin (SIGMA Co., USA).

We used the MTT assay to determine the percentage of viable cells. After cultivation was completed, a solution of MTT was added to the cultivation solution and the mixture was incubated at 37° C. for 4 hr. After incubation, culture medium was thoroughly removed, and the residue was treated with DMSO (Dimethyl sulfoxide) and shaken for 20 min. The optical density at 580 nm was measured by an ELISA microplate reader. The results are shown in the Table 2.

TABLE 2
Effects of Morus alba extract, oxyresveratrol, and mulberroside
A on cell viability and melanin production of murine melanoma
B16 cells

| | Concentration | Melanin | |
|---|---|---|---|
| Compounds | (μg/mL) | production (%) | Cell viability (%) |
| Arbutin | 1 | 99.4 ± 2.1 | 102.4 ± 5.3 |
| | 5 | 100.8 ± 4.5 | 100.1 ± 7.8 |
| | 10 | 103.2 ± 1.9 | 98.5 ± 6.3 |
| | 50 | 98.9 ± 8.2 | 96.6 ± 8.0 |
| | 100 | 98.2 ± 6.9 | 93.2 ± 5.6 |
| Kojic acid | 1 | 102.4 ± 4.7 | 95.2 ± 8.3 |
| | 5 | 98.2 ± 7.1 | 97.2 ± 7.6 |
| | 10 | 96.5 ± 3.7 | 96.2 ± 5.0 |
| | 50 | 94.1 ± 5.2 | 86.7 ± 3.4 |
| | 100 | 91.6 ± 7.1 | 81.7 ± 8.5 |
| Morus alba Extract | 1 | 97.2 ± 5.3 | 105.6 ± 1.3 |
| | 5 | 93.3 ± 9.2 | 103.0 ± 2.6 |
| | 10 | 90.4 ± 6.3 | 98.7 ± 6.9 |
| | 50 | 64.2 ± 7.6 | 96.1 ± 7.5 |
| | 100 | 23.1 ± 7.2 | 94.5 ± 8.1 |
| Oxyresveratrol | 1 | 94.3 ± 7.7 | 99.5 ± 5.4 |
| | 5 | 90.8 ± 4.4 | 97.4 ± 5.9 |
| | 10 | 87.5 ± 6.9 | 95.3 ± 7.6 |
| | 50 | 57.3 ± 8.1 | 66.2 ± 8.3 |
| | 100 | 19.5 ± 8.2 | 23.1 ± 4.8 |
| Mulberroside A | 1 | 98.3 ± 1.0 | 103.2 ± 1.5 |
| | 5 | 92.4 ± 2.1 | 101.1 ± 3.8 |
| | 10 | 91.2 ± 7.3 | 98.7 ± 8.4 |
| | 50 | 59.8 ± 9.0 | 97.1 ± 5.9 |
| | 100 | 21.8 ± 5.4 | 94.0 ± 6.4 |

Melan-a

We assayed the agents of the present invention as well as the reference compounds arbutin and kojic acid to determine their effects on melanogenesis and cell viability in the melan-a cell line. Melan-a cells were grown in 10 mL of RPMI 1640 medium supplemented with antibiotics, 10% fetal bovine serum (FBS), and 20 nM TPA. The cell suspension was inoculated in a 24-well plate (10⁵ cells/mL) and the cells were allowed to completely adhere to the plate for 24 hr. Test samples dissolved in DMSO were added to the plate and incubated at 37° C. for 72 hr in a CO₂ incubator. The culture medium was then thoroughly removed and the cells were isolated through trypsin treatment. The suspensions were then centrifuged for 5 min to collect cells, which were then treated with 5% trichloroacetic acid (TCA), stirred and centrifuged to precipitate the melanin. Melanin was washed with PBS and dissolved in 1 N NaOH. Absorbance at 475 nm was measured. Melanin concentration was determined from a standard concentration curve of synthetic melanin (SIGMA Co., USA).

We used the MTT assay to determine the percentage of viable cells. After cultivation was completed, a solution of MTT was added to the cultivation solution and the mixture was incubated at 37° C. for 4 hr. After incubation, culture medium was thoroughly removed, and the residue was treated with DMSO (Dimethyl sulfoxide) and shaken for 20 min. The optical density at 580 nm was measured by an ELISA microplate reader. The results are shown in the Table 3.

TABLE 3
Effects of Morus alba extract, oxyresveratrol, and mulberroside A
on cell viability and melanin production of melan-a cell line.

| | Concentration | Melanin | Cell viability |
|---|---|---|---|
| Compounds | (μg/mL) | production (%) | (%) |
| Arbutin | 1 | 104.2 ± 3.2 | 104.2 ± 7.2 |
| | 5 | 103.7 ± 4.6 | 105.2 ± 8.3 |
| | 10 | 102.0 ± 5.7 | 100.7 ± 5.0 |
| | 50 | 100.4 ± 4.0 | 98.3 ± 7.7 |
| | 100 | 99.5 ± 7.2 | 93.8 ± 5.6 |
| Kojic acid | 1 | 103.4 ± 5.4 | 95.3 ± 8.0 |
| | 5 | 100.5 ± 8.2 | 96.8 ± 4.7 |
| | 10 | 99.4 ± 7.1 | 95.1 ± 9.0 |
| | 50 | 95.1 ± 6.7 | 84.2 ± 8.2 |
| | 100 | 92.4 ± 8.0 | 80.5 ± 8.3 |
| Morus alba Extract | 1 | 98.0 ± 3.2 | 103.8 ± 2.8 |
| | 5 | 94.2 ± 5.3 | 102.1 ± 6.1 |
| | 10 | 90.1 ± 7.1 | 99.4 ± 7.4 |
| | 50 | 68.3 ± 8.9 | 96.9 ± 8.2 |
| | 100 | 24.9 ± 6.8 | 95.2 ± 6.9 |
| Oxyresveratrol | 1 | 95.1 ± 3.1 | 99.0 ± 5.8 |
| | 5 | 90.2 ± 6.4 | 96.9 ± 7.9 |
| | 10 | 88.5 ± 7.4 | 94.7 ± 4.2 |
| | 50 | 60.3 ± 9.1 | 68.5 ± 5.5 |
| | 100 | 20.6 ± 4.9 | 27.3 ± 7.8 |
| Mulberroside A | 1 | 98.5 ± 7.2 | 100.1 ± 6.0 |
| | 5 | 93.6 ± 4.8 | 99.2 ± 7.6 |
| | 10 | 92.8 ± 6.5 | 99.0 ± 9.2 |
| | 50 | 61.2 ± 8.0 | 97.3 ± 8.4 |
| | 100 | 23.7 ± 3.8 | 95.4 ± 7.3 |

As evidence by the data in Tables 2 and 3, treatment of melanoma B-16 and melan-a cells with the agents of the present invention resulted in significant reductions of melanogenesis in these cells. Treatment of these cells with 100 μg/mL Morus alba extract resulted in a 76.9% reduction in melanin production in melanoma B-16 cells and a 75.1% reduction in melanin production in melan-a cells. Treatment of these cells with 100 μg/mL oxyresveratrol resulted in an 80.5% reduction in melanin production in melanoma B-16 cells and a 79.4% reduction in melanin production in melan-a cells. Treatment of these cells with 100 μg/mL mulberroside A resulted in a 78.2% reduction in melanin production in melanoma B-16 cells and a 76.3% reduction in melanin production in melan-a cells. Kojic acid and arbutin did not show inhibitory effects on melanin production at any of the concentrations assayed. The pigmentation of a confluent monolayer of melanocytes is visible microscopically, as the cells contain black melanin pigment. Reduced pigmentation was observed in the cells treated with Morus alba extract, oxyresveratrol and mulberroside A.

Extended Duration of Tyrosinase Inhibition

Inhibitory Effects on Mushroom Tyrosinase

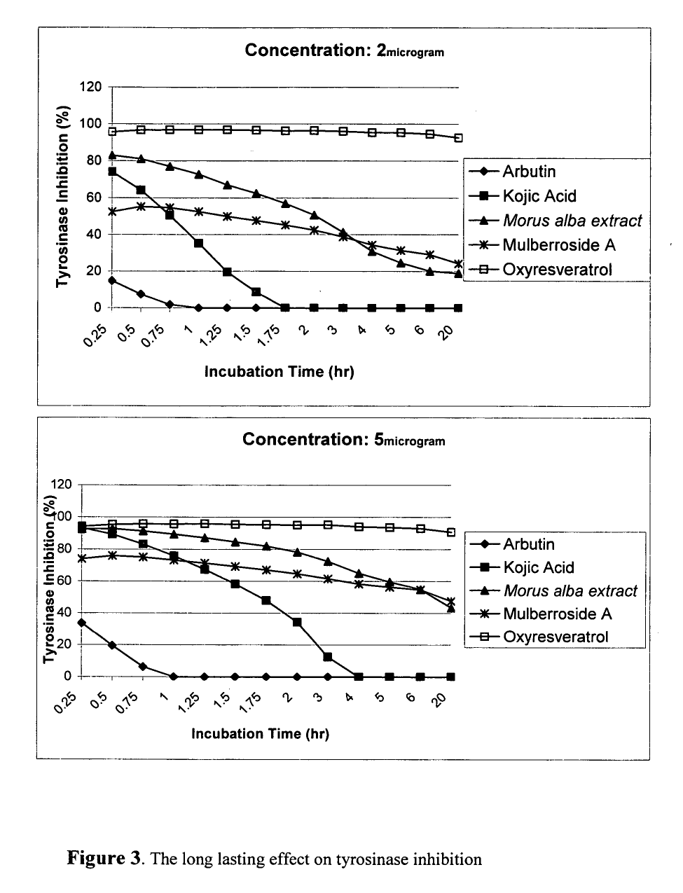

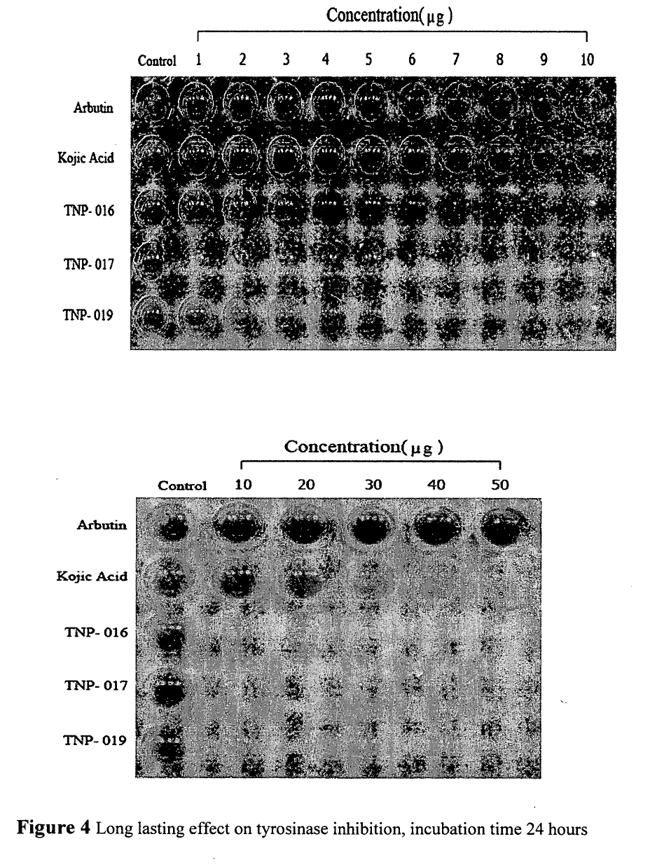

We assayed the agents of the present invention as well as the reference compounds arbutin and kojic acid to determine the duration of their inhibitory activity on tyrosinase. Using the experimental conditions described in the Mushroom Tyrosinase experiment described above, the reaction mixtures, containing various concentrations from 1 μg/mL to 50 μg/mL of the agents assayed, were incubated at 37° C. and spectrophotometric measurements of dopachrome were made at time intervals of up to 24 hours. The results are shown in FIGS. 3 and 4.

Most inhibitors of melanogenesis lose activity after one or two hours of reaction time. As shown in FIG. 3, the reference compound arbutin loses activity in about one hour at either 2 μg/mL or 5 μg/mL. Moreover, the reference compound kojic acid loses activity within two hours at 2 μg/mL and within five hours at 5 μg/mL. The active agents of the present invention, however, possess significant inhibitory activity on tyrosinase even up to 24 hours of reaction time.

The inhibitory effects of the agents assayed on tyrosinase activity were also assessed upon incubation of the reaction mixtures for 24 hours, as shown in FIG. 4. Oxyresveratrol exhibited about 90% inhibition of tyrosinase activity after 24 hours even at the lower concentrations tested. Kojic acid exhibited an IC₅₀ value for tyrosinase inhibition of 241 μM, while the respective IC₅₀ values for oxyresveratrol and mulberroside A were 2.6 μM and 20 μM. In view of these IC₅₀ values, oxyresveratrol exhibited a 92-fold stronger inhibitory effect on tyrosinase activity than kojic acid, while mulberroside A showed a 12-fold stronger activity than kojic acid.

RT-PCR Analysis of Tyrosinase mRNA:

To elucidate the mechanism by which Morus alba extract and mulberroside A exerts its effect, RT-PCR analysis was performed. mRNA from cultured murine melanoma B-16 cells were isolated using a Promega polytract system 1000 mRNA isolation kit. Approximately 80 to 100 μg of mRNA was isolated from 10⁸ cells. We synthesized the 3′ primer 5′GACCTCAGTTCCCCTTCAAA3′ (nucleotides 197-216 from the ATG start codon) and the 5′ primer 5′TCTCATCCCCAGTTAGTTCT3′ (nucleotides 669-688 from the ATG start codon) for use in the RT-PCR analysis of tyrosinase mRNA.

The reaction mixture for reverse transcription of mRNA had a total volume of 20 μL and contained 20 ng of mRNA, 2 μL of 10× reverse transcription buffer, 2 μL of 10 mM MnCl₂, 0.2 mM of each of the dNTPs, 50 pmole of the 3′ primer and 5 U of rTth DNA polymerase (Perkin Elmer), an enzyme that reverse transcribes RNA in the presence of Mn²⁺. This mixture was incubated for 1 minute at 94° C., then for 30 seconds at 55° C. and finally for 10 minutes at 72° C.

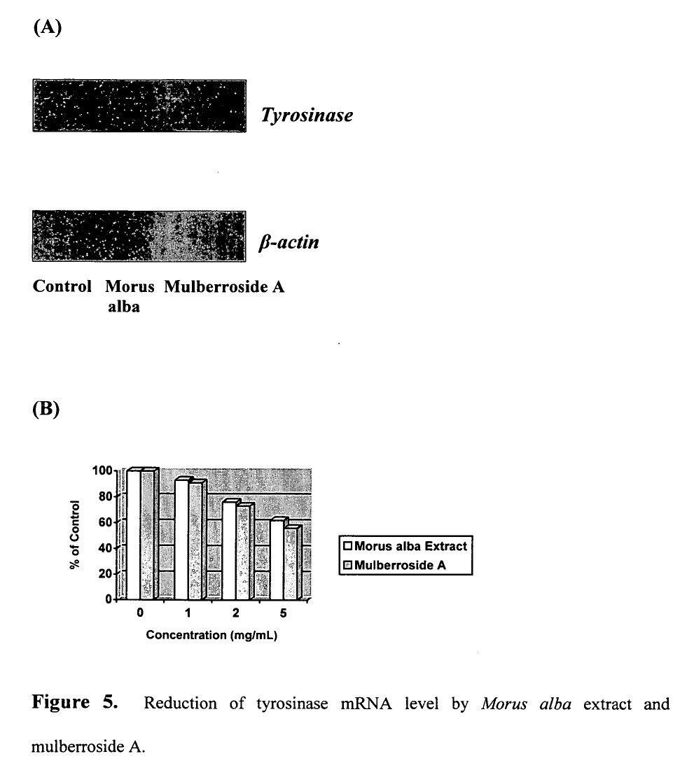

For PCR amplification of cDNA, 80 μL of a PCR mixture containing 8 μL of 10× chelating buffer, 1 mM MgCl2 and 50 pmole of 5′ primer was added to the reverse transcription reaction mixture. DNA amplification was performed on a Perkin Elmer Gene Amp PCR system 2400 thermal cycler. The PCR cycle conditions included melting for 30 sec at 94° C., annealing for 30 sec at 55° C., and extension for 1 min at 72° C. The PCR ran for 28 cycles, and the products (492 bp) were analyzed by 2% agarose gel electrophoresis. As an internal control, mouse β-actin mRNA was also amplified using the mouse β-actin amplimer set (Clontech, USA). The results are shown in FIG. 5.

Morus alba extract and mulberroside A both decreased the amount of tyrosinase mRNA in a dose-dependent manner. Thus, both Morus alba extract and mulberroside A downregulate the production of tyrosinase mRNA with consequent inhibition of tyrosinase activity.

Cosmetic compositions of the present invention are described more fully by way of the following non-limiting examples of cosmetically acceptable vehicles. Morus alba extract, oxyresveratrol enriched Morus alba extract and mulberrocide a enriched extract, oxyresveratrol, and mulberrocide A may each be used to prepare cosmetic compositions of the invention, such as skin softener (skin lotion), nutrient emulsion (milk lotion), nutrient cream, massage cream, essense and facial pack. The amount of extract or purified compound added is about 0.000015% (w/w), preferably 0.001˜1% (w/w) based upon the dry weight of each.

### EXAMPLE 1

An exemplary formula of a skin softener containing Morus alba extract of the present invention is shown in Table 4.

TABLE 4

| Components | Content (%) |
|---|---|
| Morus alba extract | 0.01 |
| 1,3-butylene glycol | 6.0 |
| Sodium hyaluronate | 2.0 |
| Glycerin | 4.0 |
| Oleyl alcohol | 0.1 |
| Polysorbate 20 | 0.5 |
| Ethanol | 15.0 |
| Preservatives | Proper amount |
| Benzophenone-9 | 0.05 |
| Fragrance | Proper amount |
| Distilled water | Remainder |
| Total | 100.0 |

### EXAMPLE 2

An exemplary formula of a milk lotion containing Morus alba extract of the present invention is shown in Table 5.

TABLE 5

| Components | Content (%) |
|---|---|
| Morus alba extract | 0.1 |
| 1,3-butylene glycol | 6.0 |
| Stearic acid | 0.4 |
| Glycerin | 4.0 |
| Cetostearyl alcohol | 1.2 |
| Polysorbate 60 | 1.5 |
| Glyceryl Stearate | 1.0 |
| Triethanolamine | 0.25 |
| Tocopheryl acetate | 3.0 |
| Liquid paraffin | 5.0 |
| Squalane | 3.0 |
| Macadamia nut oil | 2.0 |
| Sorbitan sesquioleate | 0.6 |
| Carboxy vinyl polymer | 0.15 |
| Preservatives | Proper amount |
| Benzophenone-9 | 0.05 |
| Fragrance | Proper amount |
| Distilled water | Remainder |
| Total | 100.0 |

### EXAMPLE 3

An exemplary formula of a nutrient cream containing Morus alba extract of the present invention is shown in Table 6.

TABLE 6

| Components | Content (%) |
|---|---|
| Morus alba extract | 0.5 |
| 1,3-butylene glycol | 6.0 |
| Stearic acid | 1.5 |
| Glycerin | 4.0 |
| Cetostearyl alcohol | 2.5 |
| Polysorbate 60 | 1.5 |
| Glyceryl stearate | 2.0 |
| Triethanolamine | 0.5 |
| Tocopheryl acetate | 0.1 |
| Liquid paraffin | 10.0 |
| Squalane | 3.0 |
| Vaselin | 7.0 |
| Sorbitan sesquioleate | 0.8 |
| Wax | 2.0 |
| Preservatives | Proper amount |
| Benzophenone-9 | 0.01 |
| Fragrance | Proper amount |
| Distilled water | Remainder |
| Total | 100.0 |

### EXAMPLE 4

An exemplary formula of an essence containing Morus alba extract of the present invention is shown in Table 7.

TABLE 7

| Components | Content (%) |
|---|---|
| Morus alba extract | 0.5 |
| Allantoin | 0.1 |
| Panthenol | 0.3 |
| Glycerin | 10.0 |
| EDTA | 0.02 |
| Hydroxy ethyl cellulose | 0.1 |
| Sodium hyaluronate | 8.0 |
| Triethanolamine | 0.18 |
| Carboxy vinyl polymer | 0.2 |
| Octyldodeces-25 | 0.6 |
| Ethanol | 6.0 |
| Pigment | Proper amount |
| Preservatives | Proper amount |
| Benzophenone-9 | 0.04 |
| Fragrance | Proper amount |
| Distilled water | Remainder |
| Total | 100.0 |

### EXAMPLE 5

An exemplary formula of a massage cream containing Morus alba extract of the present invention is shown in Table 8.

TABLE 8

| Components | Content (%) |
|---|---|
| Morus alba extract | 0.02 |
| Sorbitan stearate | 0.6 |
| Stearic acid | 1.0 |
| Glycerin | 6.0 |
| Cetostearyl alcohol | 2.5 |
| Polysorbate 60 | 1.5 |
| Glyceryl stearate | 2.0 |
| Triethanolamine | 0.5 |
| Isostearyl isostearate | 5.0 |
| Mineral oil | 35.0 |
| Squalane | 5.0 |
| Dimethicone | 1.0 |
| Xanthan gum | 0.1 |
| Hydroxyethyl cellulose | 0.12 |
| Preservatives | Proper amount |
| Pigment | Proper amount |
| Fragrance | Proper amount |
| Distilled water | Remainder |
| Total | 100.0 |

### EXAMPLE 6

An exemplary formula of a facial pack cream containing Morus alba extract of the present invention is shown in Table 9.

TABLE 9

| Components | Content (%) |
|---|---|
| Morus alba extract | 0.01 |
| Allantoin | 0.1 |
| Panthenol | 0.4 |
| Glycerin | 3.0 |
| Polyvinyl alcohol | 15.0 |
| PEG-40 hydrogenated castor oil | 0.3 |
| PEG 1500 | 2.0 |
| Cellulose gum | 0.15 |
| Ethanol | 6.0 |
| Pigment | Proper amount |
| Preservatives | Proper amount |
| Benzophenone-9 | 0.04 |
| Fragrance | Proper amount |
| Distilled water | Remainder |
| Total | 100.0 |

### EXAMPLE 7

An exemplary formula of a skin softener containing oxyresveratrol of the present invention is shown in Table 10.

TABLE 10

| Components | Content (%) |
|---|---|
| Oxyresveratrol | 0.001 |
| 1,3-butylene glycol | 6.0 |
| Sodium hyaluronate | 2.0 |
| Glycerin | 4.0 |
| Oleyl alcohol | 0.1 |
| Polysorbate 20 | 0.5 |
| Ethanol | 15.0 |
| Preservatives | Proper amount |
| Benzophenone-9 | 0.05 |
| Fragrance | Proper amount |
| Distilled water | Remainder |
| Total | 100.0 |

### EXAMPLE 8

An exemplary formula of a milk lotion containing oxyresveratrol of the present invention is shown in Table 11.

TABLE 11

| Components | Content (%) |
|---|---|
| Oxyresveratrol | 0.005 |
| 1,3-butylene glycol | 6.0 |
| Stearic acid | 0.4 |
| Glycerin | 4.0 |
| Cetostearyl alcohol | 1.2 |
| Polysorbate 60 | 1.5 |
| Glyceryl Stearate | 1.0 |
| Triethanolamine | 0.25 |
| Tocopheryl acetate | 3.0 |
| Liquid paraffin | 5.0 |
| Squalane | 3.0 |
| Macadamia nut oil | 2.0 |
| Sorbitan sesquioleate | 0.6 |
| Carboxy vinyl polymer | 0.15 |
| Preservatives | Proper amount |
| Benzophenone-9 | 0.05 |
| Fragrance | Proper amount |
| Distilled water | Remainder |
| Total | 100.0 |

### EXAMPLE 9

An exemplary formula of a nutrient cream containing oxyresveratrol of the present invention is shown in Table 12.

TABLE 12

| Components | Content (%) |
|---|---|
| Oxyresveratrol | 0.01 |
| 1,3-butylene glycol | 6.0 |
| Stearic acid | 1.5 |
| Glycerin | 4.0 |
| Cetostearyl alcohol | 2.5 |
| Polysorbate 60 | 1.5 |
| Glyceryl stearate | 2.0 |
| Triethanolamine | 0.5 |
| Tocopheryl acetate | 0.1 |
| Liquid paraffin | 10.0 |
| Squalane | 3.0 |
| Vaselin | 7.0 |
| Sorbitan sesquioleate | 0.8 |
| Wax | 2.0 |
| Preservatives | Proper amount |
| Benzophenone-9 | 0.01 |
| Fragrance | Proper amount |
| Distilled water | Remainder |
| Total | 100.0 |

### EXAMPLE 10

An exemplary formula of an essence containing oxyresveratrol of the present invention is shown in Table 13.

TABLE 13

| Components | Content (%) |
|---|---|
| Oxyresveratrol | 0.01 |
| Allantoin | 0.1 |
| Panthenol | 0.3 |
| Glycerin | 10.0 |
| EDTA | 0.02 |
| Hydroxy ethyl cellulose | 0.1 |
| Sodium hyaluronate | 8.0 |
| Triethanolamine | 0.18 |
| Carboxy vinyl polymer | 0.2 |
| Octyldodeces-25 | 0.6 |
| Ethanol | 6.0 |
| Pigment | Proper amount |
| Preservatives | Proper amount |
| Benzophenone-9 | 0.04 |
| Fragrance | Proper amount |
| Distilled water | Remainder |
| Total | 100.0 |

### EXAMPLE 11

An exemplary formula of a massage cream containing oxyresveratrol of the present invention is shown in Table 14.

TABLE 14

| Components | Content (%) |
|---|---|
| Oxyresveratrol | 0.002 |
| Sorbitan stearate | 0.6 |
| Stearic acid | 1.0 |
| Glycerin | 6.0 |
| Cetostearyl alcohol | 2.5 |
| Polysorbate 60 | 1.5 |
| Glyceryl stearate | 2.0 |
| Triethanolamine | 0.5 |
| Isostearyl isostearate | 5.0 |
| Mineral oil | 35.0 |
| Squalane | 5.0 |
| Dimethicone | 1.0 |
| Xanthan gum | 0.1 |
| Hydroxyethyl cellulose | 0.12 |
| Preservatives | Proper amount |
| Pigment | Proper amount |
| Fragrance | Proper amount |
| Distilled water | Remainder |
| Total | 100.0 |

### EXAMPLE 12

An exemplary formula of a facial pack cream containing oxyresveratrol of the present invention is shown in Table 15.

TABLE 15

| Components | Content (%) |
|---|---|
| Oxyresveratrol | 0.001 |
| Allantoin | 0.1 |
| Panthenol | 0.4 |
| Glycerin | 3.0 |
| Polyvinyl alcohol | 15.0 |
| PEG-40 hydrogenated castor oil | 0.3 |
| PEG 1500 | 2.0 |
| Cellulose gum | 0.15 |
| Ethanol | 6.0 |
| Pigment | Proper amount |
| Preservatives | Proper amount |
| Benzophenone-9 | 0.04 |
| Fragrance | Proper amount |
| Distilled water | Remainder |
| Total | 100.0 |

### EXAMPLE 13

An exemplary formula of a skin softener containing mulberroside A of the present invention is shown in Table 16.

TABLE 16

| Components | Content (%) |
|---|---|
| Mulberroside A | 0.005 |
| 1,3-butylene glycol | 6.0 |
| Sodium hyaluronate | 2.0 |
| Glycerin | 4.0 |
| Oleyl alcohol | 0.1 |
| Polysorbate 20 | 0.5 |
| Ethanol | 15.0 |
| Preservatives | Proper amount |
| Benzophenone-9 | 0.05 |
| Fragrance | Proper amount |
| Distilled water | Remainder |
| Total | 100.0 |

### EXAMPLE 14

An exemplary formula of a milk lotion containing mulberroside A of the present invention is shown in Table 17.

TABLE 17

| Components | Content (%) |
|---|---|
| Mulberroside A | 0.02 |
| 1,3-butylene glycol | 6.0 |
| Stearic acid | 0.4 |
| Glycerin | 4.0 |
| Cetostearyl alcohol | 1.2 |
| Polysorbate 60 | 1.5 |
| Glyceryl Stearate | 1.0 |
| Triethanolamine | 0.25 |
| Tocopheryl acetate | 3.0 |
| Liquid paraffin | 5.0 |
| Squalane | 3.0 |
| Macadamia nut oil | 2.0 |
| Sorbitan sesquioleate | 0.6 |
| Carboxy vinyl polymer | 0.15 |
| Preservatives | Proper amount |
| Benzophenone-9 | 0.05 |
| Fragrance | Proper amount |
| Distilled water | Remainder |
| Total | 100.0 |

### EXAMPLE 15

An exemplary formula of a nutrient cream containing mulberroside A of the present invention is shown in Table 18.

TABLE 18

| Components | Content (%) |
|---|---|
| Mulberroside A | 0.05 |
| 1,3-butylene glycol | 6.0 |
| Stearic acid | 1.5 |
| Glycerin | 4.0 |
| Cetostearyl alcohol | 2.5 |
| Polysorbate 60 | 1.5 |
| Glyceryl stearate | 2.0 |
| Triethanolamine | 0.5 |
| Tocopheryl acetate | 0.1 |
| Liquid paraffin | 10.0 |
| Squalane | 3.0 |
| Vaselin | 7.0 |
| Sorbitan sesquioleate | 0.8 |
| Wax | 2.0 |
| Preservatives | Proper amount |
| Benzophenone-9 | 0.01 |
| Fragrance | Proper amount |
| Distilled water | Remainder |
| Total | 100.0 |

### EXAMPLE 16

An exemplary formula of an essence containing mulberroside A of the present invention is shown in Table 19.

TABLE 19

| Components | Content (%) |
|---|---|
| Mulberroside A | 0.02 |
| Allantoin | 0.1 |
| Panthenol | 0.3 |
| Glycerin | 10.0 |
| EDTA | 0.02 |
| Hydroxy ethyl cellulose | 0.1 |
| Sodium hyaluronate | 8.0 |
| Triethanolamine | 0.18 |
| Carboxy vinyl polymer | 0.2 |
| Octyldodeces-25 | 0.6 |
| Ethanol | 6.0 |
| Pigment | Proper amount |
| Preservatives | Proper amount |
| Benzophenone-9 | 0.04 |
| Fragrance | Proper amount |
| Distilled water | Remainder |
| Total | 100.0 |

### EXAMPLE 17

An exemplary formula of a massage cream containing mulberroside A of the present invention is shown in Table 20.

TABLE 20

| Components | Content (%) |
|---|---|
| Mulberroside A | 0.002 |
| Sorbitan stearate | 0.6 |
| Stearic acid | 1.0 |
| Glycerin | 6.0 |
| Cetostearyl alcohol | 2.5 |
| Polysorbate 60 | 1.5 |
| Glyceryl stearate | 2.0 |
| Triethanolamine | 0.5 |
| Isostearyl isostearate | 5.0 |
| Mineral oil | 35.0 |
| Squalane | 5.0 |
| Dimethicone | 1.0 |
| Xanthan gum | 0.1 |
| Hydroxyethyl cellulose | 0.12 |
| Preservatives | Proper amount |
| Pigment | Proper amount |
| Fragrance | Proper amount |
| Distilled water | Remainder |
| Total | 100.0 |

### EXAMPLE 18

An exemplary formula of a facial pack cream containing mulberroside A of the present invention is shown in Table 21.

| Components | Content (%) |
|---|---|
| Mulberroside A | 0.002 |
| Allantoin | 0.1 |
| Panthenol | 0.4 |
| Glycerin | 3.0 |
| Polyvinyl alcohol | 15.0 |
| PEG-40 hydrogenated castor oil | 0.3 |
| PEG 1500 | 2.0 |
| Cellulose gum | 0.15 |
| Ethanol | 6.0 |
| Pigment | Proper amount |
| Preservatives | Proper amount |
| Benzophenone-9 | 0.04 |
| Fragrance | Proper amount |
| Distilled water | Remainder |
| Total | 100.0 |

We claim:

1. A tyrosinase inhibitor composition comprising an extract of Morus alba.

2. A cosmetic composition comprising a tyrosinase inhibitor composition according to claim 1 in a cosmetically acceptable vehicle.

3. A cosmetic composition according to claim 2 wherein the concentraion of Morus alba extract is about 0.00001˜5% (w/w).

4. A method for use of the tyrosinase inhibitor composition of claim 1 to inhibit the production of melanin in a subject comprising contacting the subject with a therapeutically effective amount of the tyrosinase inhibitor composition of claim 1.

5. The method of claim 4 wherein the subject is a human.

6. A tyrosinase inhibitor composition comprising oxyresveratrol.

7. A cosmetic composition comprising a tyrosinase inhibitor composition according to claim 6 in a cosmetically accepable vehicle.

8. A cosmetic composition according to claim 7 wherein the concentration of oxyresveratrol is about 0.00001˜5% (w/w).

9. A method for use of the tyrosinase inhibitor composition of claim 6 to inhibit the production of melanin in a subject comprising contacting the subject with a therapeutically effective amount of the tyrosinase inhibitor composition of claim 6.

10. The method of claim 9 wherein the subject is a human

11. A tyrosinase inhibitor composition comprising mulberroside A.

12. A cosmetic composition comprising a tyrosinase inhibitor composition according to claim 11 in a cosmetically acceptable vehicle.

13. A cosmetic composition according to claim 12 wherein the concentration of mulberrocide A is about 0.00001˜5% (w/w).

14. A method for use of the tyrosinase inhibitor composition of claim 11 to inhibit the production of melanin in a subject comprising contacting the subject with a therapeutically effective amount of the tyrosinase inhibitor composition of claim 11.

15. The method of claim 14 wherein the subject is a human.

16. A method for the isolation of a tyrosinase inhibitor composition from Morus alba, the method comprising extraction of the Morus alba with approximately 30-100% ethanol:water to obtain the tyrosinase inhibitor composition.

17. The method of claim 16 further comprising subjecting the tyrosinase inhibitor composition to fractionation via column chromatography or solvent partitioning to obtain an enriched tyrosinase inhibitor composition.
